(19) United States
(12) Patent Application Publication
(10) Pub. No.: US 2015/0027277 A1
(43) Pub. Date: Jan. 29, 2015

(54) RATCHET AND METHOD OF PRODUCING SAME, AS WELL AS A TORQUE TRANSMISSION SYSTEM AND METHOD FOR TRANSMITTING TORQUE TO AN IMPLANT DRIVER, AND USE OF SUCH RATCHET IN THE MEDICAL FIELD

(71) Applicant: Hipp medical AG, Kolbingen (DE)

(72) Inventor: Markus Waizenegger, Kolbingen (DE)

(21) Appl. No.: 14/373,142

(22) PCT Filed: Jan. 14, 2013
(86) PCT No.: PCT/EP2013/000093

§ 371 Date: Jul. 18, 2014

(30) Foreign Application Priority Data
Feb. 9, 2012 (DE) 102012101050.1

(51) Int. Cl. A61C 1/18; A61C 8/00; A61B 19/00; B25B 13/46; A61B 17/88

(52) U.S. Cl. CPC A61C 1/186; B25B 13/46; A61B 17/8875; A61B 19/30; A61C 8/0089; A61B 2017/00526
USPC 81/60; 83/13

(57) ABSTRACT

A ratchet (1) is provided, in particular a ratchet for surgical interventions or a dental ratchet, wherein the ratchet has a handle (2) and a pivot housing (4) arranged thereon, said pivot housing being a flat spiral (6). The spiral (6) is flexible and can be actuated such that the spiral (6) coils up in a plane that is oriented substantially vertically to the pivot axis D. The invention also relates to a torque transmission system which comprises a ratchet (1) and an implant driver. The implant driver has a first portion that is cylindrical and a second portion that is cylindrical or frustoconical, the second portion additionally having a receptacle for a medical tool, a surgical implant or any other object. In this way, an improved ratchet is obtained with which the torque to be applied can be applied to the implant driver in a metered manner.

### FIELD

The invention relates to a ratchet according to the preamble of claim 1, in particular a ratchet for surgical interventions or a dental ratchet. The invention further relates to a torque transmission system according to claim 17 and to the use of such a ratchet in the medical field according to claim 22. Moreover, in accordance with claim 23 the invention relates to a method for transmitting a torque to be applied with such a ratchet to an implant driver, and to a method for manufacturing such a ratchet according to claim 27.

When performing surgical interventions using tools and/or medical equipment, the problem of sufficient sterilization is well-known. In order to prevent blood or tissue residues and other wound excretions from getting into slits or junctures of correlating components and from depositing there, value is attached in the medical field to the fact that the tools as used are composed of as few individual components as possible.

By forming the tools of as few components as possible, in particular in one piece, it is possible to largely reduce infections that may be caused by deposits or germs positioned at the junctures of tools formed of several components.

Furthermore, the required sterilization of single-piece medical tools does not necessitate a dismounting of individual components which may result in possible abrasion and signs of wear at the places of direct contact.

Tools consisting of few individual pieces or being formed of just one piece have the further advantage that an incorrect assembly by the user may be reduced or avoided, in particular also if several tools of identical construction are cleaned in one container.

Moreover, the effort resulting from the individual components of multi-piece tools being assembled and from the operativeness of the assembled tool subsequently being tested may be reduced.

Thus, while tools consisting of very few pieces, frequently only of one single piece, for instance, single-piece forceps, saws, drills and the like, are preferably used in many medical fields, it is still the case in oral, jaw and facial surgery that some special tools have to be constructed of several pieces to be able to fulfil the desired function.

This is of particular importance above all in the field of dental and implantation treatment since, when implant carriers or tooth bases are inserted in the jaw, particular attention has to be taken that no infectious agents are transferred which could not just attack the gingiva, but possibly even the jaw bone. Such implant carriers or tooth bases are usually screwed into the jaw bone by means of dental ratchets formed of several pieces.

In order to avoid the above-mentioned disadvantages, in particular the infection risk associated with the multi-piece design of medical tools, tools preferably manufactured of one piece, i.e. single-piece dental ratchets, should also be used in the field of oral, jaw and facial surgery.

WO 2006/029542 A1 discloses a torque wrench for the medical field which is designed as a ratchet instrument. At the periphery of the receiving opening a restrictedly movable latch segment is arranged whose front part points to the receiving opening. On actuation of the torque wrench in forward direction, the torque to be generated is applied via a deflectable linear elastic bending branch by means of a forward force exerted by the user.

A latch spring extends from the latch segment into a collar region. The latch segment and the latch spring form a latch composed of several pieces which may alternatively also be designed as one piece. The latch segment and the latch spring are arranged in a channel-shaped clearance allowing the deflection of both of them in the plane against the force of the latch spring.

In the field of oral, jaw and facial surgery it is often important to transmit the torque to be transmitted from the dental ratchet to the implant driver in a sensitive manner. The torque wrench of WO 2006/029542 A1, however, does not allow for this since the torque transmission takes only place when the latch segment gets into contact with the upper first stop. Thus, a sensitive transmission of the torque to the implant driver is not possible.

DE 20 2007 015 689 U1 proposes a dental ratchet in which a so-called mono ratchet head is arranged on the handle. The mono ratchet head provides the enclosure for the implant driver as a spring element with a latch element. The flexible enclosure is further designed to be annular with an interruption or a gap, respectively, so that it has the shape of an open ring. A latch element is formed on a free end of the enclosure or the open ring, respectively. The latch element allows for the movement of the implant driver in one direction only. The other direction of rotation is achieved by latching the implant driver into the latch element.

Another essential component of the dental ratchet of DE 20 2007 015 689 U1 is the groove-shaped contour design of the implant driver such as it exists, for instance, also with a torx. Only this design makes it possible to achieve the desired functioning with the ratchet of DE 20 2007 015 689 U1.

A metered transmission of the torque to the implant driver is not possible, either, with the dental ratchet of DE 20 2007 015 689 U1.

Accordingly, it is an object of the present invention to provide a ratchet by means of which the torque to be applied can be applied to an implant driver in a precisely metered manner

This object is solved by a ratchet with the features of claim 1.

In accordance with the invention there is proposed a ratchet comprising a handle and a pivot housing arranged thereon, said pivot housing being a flat spiral. Furthermore, the spiral is flexible and the ratchet can be actuated such that the spiral coils up in a plane that is oriented substantially vertically to the pivot axis.

The design of the pivot housing as a flexible spiral makes it possible in a simple manner that, when the ratchet is rotated in the direction of torque transmission, part of the force to be applied by a user flows into the flexible deformation of the spiral and the other part causes the torque to be transmitted to an implant driver. First of all, the force applied by the user to the handle causes a flexible deformation of the spiral until the implant driver has been enclosed in a force-fit manner by the spiral due to the friction occurring between the elements, so that subsequently the further force applied by the user causes the torque transmission to the implant driver. If the force additionally applied by the user, however, becomes too large, which might, for instance, result in a damage of the jaw, the implant carrier, the tooth base, or the like, the ratchet slips or spins with respect to the implant driver. Thus, it is possible to transmit the torque transmission to the implant driver in a metered manner without damaging the human body or other components.

The deformation of the flexible spiral is performed by coiling it up in a plane, i.e. when regarding the top view (two-dimensional), the spiral coils up about a pivot axis of the pivot housing, i.e. inwardly, during torque transmission. The flexibility of the spiral thus extends merely across one plane. When the ratchet is rotated in freewheel direction, the spiral coils away from the pivot axis, in other words it opens.

Moreover, a spring force conditioned by the designing of the pivot housing as a spiral presses the spiral with a predefined contact force additionally on the implant driver when the spiral is coiled in the direction of torque transmission. The combination of surface roughness of the spiral and contact force of the spiral on the implant driver consequently increases the torque transmission efficiently.

Furthermore, by a simple turning of the ratchet with respect to a longitudinal axis of the handle it is possible to change the direction of torque transmission, namely to a freewheeling direction prior to the process of turning. Thus, it is possible to determine the direction of torque transmission of the ratchet in an advantageous manner without taking the effort of retrofitting the ratchet.

With the configuration of the ratchet in accordance with the invention which is of simple construction it is possible to keep the manufacturing costs low.

Finally, the ratchet can be handled easily and without much effort even by less experienced personnel, for instance, by apprentices at the dentist who are made familiar with the disinfection of the tools used and with the preparation of the treatment room.

Further embodiments of the ratchet according to the invention are the subject matter of the dependent claims 2 to 16.

Thus, the ratchet may have a single-piece design. Thus, it is advantageously possible to substantially minimize the manufacturing costs of the ratchet since, besides a mounting, an assembly such as, for instance, with multi-piece ratchets, becomes superfluous. Moreover, the single-piece design of the ratchet provides a distinctly lower risk of adhering blood or tissue residues and the resulting infection risk by the formation of bacteria. The sterilization required after each use may also be performed much quicker since the ratchet need no longer be disassembled and subsequently be reassembled. The enabling of such efficient operation also relieves the strongly strained health system. Due to the single-piece configuration of the ratchet there may further be prevented advantageously that, in the case of several ratchets of identical or similar construction, the components are confused during assembly, which may possibly result in a destruction or in an impair of the functioning of the ratchet. Moreover, when the ratchet is repeatedly assembled, the delicate junctures of the components are strongly utilized and will wear. A single-piece configuration efficiently counteracts this problem.

In accordance with a further embodiment, the spiral of the ratchet may have an angle of at least 360°, preferably of at least 450°, particularly preferred of at least 630°. The angle extends from the transition from the handle to the spiral up to a free end of the spiral. The configuration of the angular dimension or of the coils of the spiral, respectively, depends on a plurality of factors. Thus, the roughness of the surface which is in operative connection with a shell surface of the implant driver may, for instance, determine the angular dimension. The higher the roughness, the smaller an angular dimension of the spiral is necessary. The flexibility of the spiral also modifies the choice of angular dimension since it determines to which degree the spiral encloses the implant driver. It is noted that the flexibility of the spiral depends on the cross-section thereof. Since the stress distribution is distributed across the entire arc length of the spiral, the stress distribution is the better the larger the angular dimension is. Since the spiral encloses the implant driver with the first coil thereof it is also possible to arrange the handle at an angle. Thus, the accessibility of the ratchet to a narrow space, such as e.g. in jaw surgery, is improved.

In a further embodiment it is possible that the spiral is designed as a logarithmic spiral, a hyperbolic spiral, or an archimedic spiral. If the spiral is designed as an archimedic spiral, the spiral may firmly enclose the implant driver since the radius of the spiral decreases proportionally to the angle from the juncture between the spiral and the handle up to the free end of the spiral. Thus, the spring force of the spiral on the implant driver is efficiently increased. Depending on the requirements of the spring force the spiral may also be designed as a logarithmic or as a hyperbolic spiral.

In accordance with a further embodiment the spiral may have at least one projection facing a pivot axis. The projection is designed on the spiral such that it may engage into a corresponding recess of an implant driver. Thus, it may be achieved that on rotation of the ratchet in the direction of torque transmission a form-fit connection is auxiliary provided in addition to the force-fit connection (friction) between the section of the spiral enclosing the implant driver and the implant driver. Furthermore, a plurality of projections may also be provided along the spiral, so that the force and/or torque transmission from the ratchet to the implant driver may be distributed more regularly on the implant driver as compared to just one projection. The torque to be transmitted is influenced by the pitch of the spiral in connection with the projection. It is noted that the corresponding recesses will accordingly have to be formed along the implant driver so as to enable a regular torque transmission. The torque to be transmitted depends on the height of the projection, the material of the ratchet and/or the hardness thereof.

In accordance with a further embodiment the projection facing the pivot axis may be arranged on a free end of the spiral. The arrangement of the projection on the end of the spiral enables an efficient combination of force-fit connection and form-fit connection since the section enclosing the implant driver ensures the force-fit connection and since the form-fit connection takes place at the end of the spiral only. Thus, the projection has to assume just the amount of force and/or torque transmission that could not be achieved by the force-fit connection (friction). The projection arranged on the end may thus be relieved advantageously, so that its wear caused by force transmission can be reduced.

Furthermore, a face of the projection which faces away from the free end of the spiral may form an angle with an edge facing the pivot axis. If the angle is an acute angle it may be achieved that the projection engages the recesses of the implant driver more firmly, i.e. hooks better, and hence the mutual latching is improved. If, in contrast, the angle is obtuse, a torque restriction may be implemented advantageously depending on the requirement since the projection disengages from the recess of the implant driver with a predetermined torque. The more obtuse the angle is designed, the lower is the torque for bending the projection away from the implant driver. The angle may optionally be designed between 45° and 150°, preferably between 70° and 120°, particularly preferred between 80° and 110°.

Moreover, the spiral may have a recess extending across a length, wherein the recess directly adjoins the face of the projection which faces away from the free end of the spiral. Thus, the free end of the spiral becomes, due to a reduced cross-section of the spiral in this region, more flexible than without a recess, which enables a restriction of torque transmission since the projection arranged on the end also deflects away from an implant driver. This makes it possible to adjust the flexibility at the free end as required. The recess may further extend across the full thickness of the spiral.

In accordance with a further embodiment the edge of the spiral facing the pivot axis may be roughened in at least one section. Such roughness may minimize or prevent the slip between the ratchet and the implant driver, so that it is achieved in an advantageous manner that the force applied by the user is transmitted to the implant driver substantially without slip. In general, roughness may be classified in three sectors. In a first sector of Raₘᵢₙ to Ra₁ a kind of mechanical adhesion exists. In this connection, a mechanical coherence of the surfaces involved, caused by a molecular interaction in the boundary surface layer exists. The surfaces are thus designed so smooth that the air available prior to their contacting will evade and thus the two surfaces involved will adhere on each other firmly. In the second sector of Ra₁ to Ra₂, which is not suited for the transmission of torque, slide friction exists. In the third sector between Ra₂ to Raₘₐₓ static friction exists in which the surface structure of the two surfaces getting into contact is designed to be so rough that they will adhere. A further, fourth sector which is larger than Raₘₐₓ may be considered as a form fit.

In a further aspect of the ratchet according to the invention the ratchet may be formed of stainless steel, titanium, ceramics, wear-proof plastics or the like. In view of a long lifetime of the ratchet, wear-proofness and/or resistance of the ratchet, in particular the clamping faces thereof, is attached particular importance to. Furthermore, the functioning may be guaranteed over a long period. Alternatively, any kinds of machining steel, construction steel or spring steel which have experienced a particular coating process so as to be protected from corrosion may also be used. Spring steel 1.4310, titanium grade 4, titanium grade 5, PEEK have turned out to be particularly suited. Furthermore, elastomers capable of being sterile come into question. It is noted that the flexibility of the spiral may be adjusted depending on the material choice.

In another aspect, the edge of the spiral facing the pivot axis may be hardened at least in one section. It is essential for the low wear of the ratchet that the ratchet, in particular the edge of the spiral facing the pivot axis, is harder than the implant driver. In this respect, different hardening methods such as, for instance, transformation hardening, grain refinement, age hardening, solid solution hardening, strain hardening, etc. may be used. It is of further advantage if the degree of hardness of the ratchet is aligned with the associated implant driver, so that the ratchet and the implant driver make up a correlating pair.

In accordance with a further embodiment the at least one projection of the spiral may be hardened in at least one section. Thus, it may be prevented efficiently that the at least one projection gets rounded and is hence no longer in accurately fitting engagement with the at least one corresponding recess. Furthermore, slip undesirably occurring by the rounding of the projection may be avoided.

In one embodiment of the ratchet according to the invention a marking may be provided in the region of the handle and/or the pivot housing and/or the spiral. This marking is particularly advantageous for the user if he or she operates at sensitive places such as, for instance, on the tissue, on wounds, in the oral region, etc., since the marking indicates in which direction the ratchet is to be rotated to tighten or to release the implant driver. Accordingly, unnecessary “trying” of the direction of rotation by the ratchet in sensitive places may effectively be avoided.

In a further embodiment of the ratchet according to the invention the handle and the pivot housing may have the same thickness. Thus, it is possible to manufacture the ratchet from a constant material thickness, which reduces the manufacturing costs and facilitates the manufacturing process significantly.

In accordance with another aspect of the present invention the handle may be cylindrical, so that ergonomic handling is enabled for the user.

In accordance with a further aspect of the ratchet according to the invention it may comprise a stop. The stop projects at least partially into the region of the pivot housing, extends preferably across a parallel plane as compared to the plane in which the spiral coils up, and is arranged on the side not facing the implant driver, so that the ratchet, when being placed on the implant driver, can only be placed on the implant driver up to the stop. When placing the ratchet on the implant driver it is accordingly prevented efficiently that the ratchet slides in axial direction of the implant driver into the tissue or into open wounds, which might possibly injure the patient.

In accordance with a further aspect of the invention, a torque transmission system is further provided in accordance with claim 17, said torque transmission system comprising a ratchet in accordance with the invention and an implant driver. Moreover, the implant driver comprises a first section of cylindrical design and a second section of cylindrical or frustoconical design, wherein the second section further comprises a receptacle for a medical tool, a surgical implant, or any other object.

By means of such a torque transmission system it is possible to transmit the force of a user via the ratchet to the implant driver in a simple manner by rotating the ratchet in the direction of torque transmission, without a loss of force and/or torque caused by slip.

A medical tool may be an Allen head, a torx head, a milling head, a polishing head, pin receptacles, pincers, a file or the like.

Advantageous further developments of the torque transmission system according to the invention are the subject matter of the dependent claims 18 to 21.

Thus, a force fit connection may exist between the ratchet and the implant driver. Torque transmission may thus be implemented easily with the aid of friction.

Alternatively or in supplement, a form-fit connection may exist between the ratchet and the implant driver. Torque transmission from the ratchet to the implant driver may thus be effected in a particularly advantageous manner.

Furthermore, the first section on the implant driver may comprise a recess arranged at least along the shell surface thereof, wherein it is achieved in the case of such a form-fit connection that the at least one projection of the spiral is in engagement with the at least one recess of the implant driver. Accordingly, the form-fit connection can be established in a simple manner

Moreover, a plurality of the recesses may be arranged along the shell surface of the implant driver with a periodic distance and/or a separation. Thus, the transmission of force from the spiral is transmitted in a particularly advantageous manner to the implant driver via at least one projection.

In accordance with a further aspect of the invention the use of a ratchet according to the invention in the field of medicine, in particular in surgery or in the dental field, for transmitting a torque to an implant driver is proposed pursuant to claim 20.

In accordance with yet another aspect of the present invention, pursuant to claim 23 there is provided a method for transmitting a torque to be applied to an implant driver by means of a ratchet according to the invention, comprising the following steps: placing the ratchet on the implant driver in the axial direction thereof, and preferably rotating the ratchet in a freewheeling direction while simultaneously lowering the ratchet along the implant driver; fixing the ratchet on the implant driver and transmitting the torque to the implant driver by rotating the ratchet in the direction of torque transmission; releasing the implant driver by rotating the ratchet positioned thereon in freewheeling direction; and detaching the ratchet from the implant driver by rotating in freewheeling direction while simultaneously removing the ratchet from the implant driver in the axial direction thereof.

Thus, the advantages and effects explained above by means of the ratchet according to the invention are equally achieved.,

Advantageous further developments of the method according to the invention are the subject matter of the dependent claims 24 to 26.

In accordance with an embodiment of the method according to the invention the steps of fixing the ratchet on the implant driver and transmitting the torque to the implant driver by rotating the ratchet in the direction of torque transmission, as well as releasing the implant driver by rotating the ratchet positioned thereon in freewheeling direction are preferably performed repeatedly, before the ratchet is detached from the implant driver.

Furthermore, the step of fixing the ratchet on the implant driver and the transmission of the torque to the implant driver by rotating the ratchet in the direction of torque transmission may be performed such that at least one projection of a spiral is in engagement with at least one recess of the implant driver. And the step of releasing the implant driver by rotating the ratchet positioned thereon in freewheeling direction may be performed such that the at least one projection of the spiral disengages from the at least one recess of the implant driver.

In accordance with a further preferred embodiment of the method according to the invention the step of fixing the ratchet on the implant driver and transmitting the torque to the implant driver by rotating the ratchet in the direction of torque transmission is performed such that a first part of the torque applied to the pivot housing continues to coil up the flexible spiral in a plane, so that a force-fit connection exists between an edge facing the pivot axis and the implant driver, and that a second part of the torque applied to the pivot housing causes a pivoting movement of the implant driver; and the step of releasing the implant driver by rotating the ratchet positioned thereon in freewheeling direction is performed such that the force-fit connection between the edge of the spiral facing the pivot axis and the implant driver is released.

It is noted that the two steps of fixing the ratchet and releasing the implant driver with the foregoing embodiment may each be combined with the corresponding steps of this embodiment.

With the method according to the invention and the use of the ratchet according to the invention it is possible to achieve in analogy the same advantages as discussed before in connection with the ratchet. For avoiding repetitions, they will not be listed here again.

In accordance with yet another aspect of the present invention, pursuant to claim 27 there is provided a method for manufacturing a ratchet, comprising the steps of: selecting a blank; and forming a contour of the ratchet by means of laser cutting, water jet cutting, wire eroding, spark eroding, or punching.

In this manner it is possible to manufacture the ratchet according to the invention in a particularly simple and precise manner and at low costs.

An exemplary advantageous further development of the method according to the invention is the subject matter of the dependent claim 28.

In accordance with this preferred embodiment of the method the blank is chosen such that it is of uniform thickness. Due to this advantageous design it is possible to manufacture the ratchet from a constant material thickness, which reduces the manufacturing costs and facilitates the manufacturing process significantly.

The afore-described features and functions of the present invention as well as further aspects and features will be described in the following by means of a detailed description of preferred embodiments under reference to the enclosed Figures. There show:

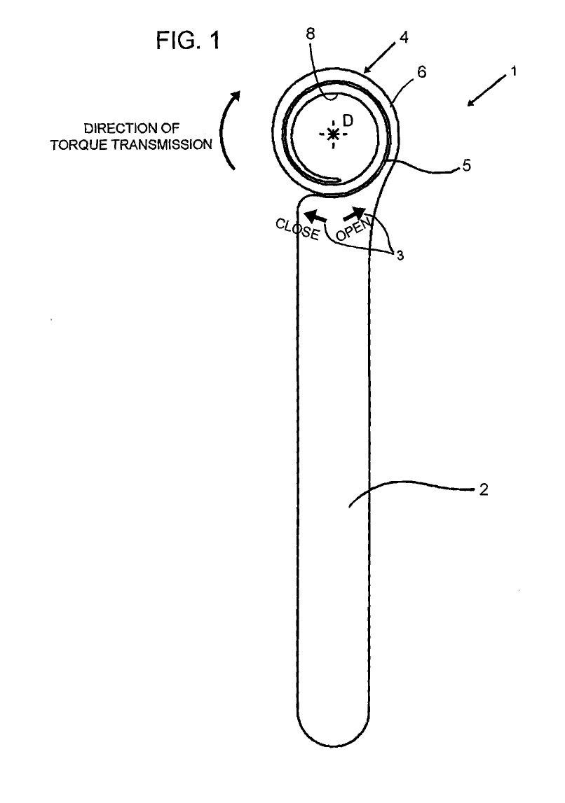

FIG. 1 a plan view of a ratchet according to the invention;

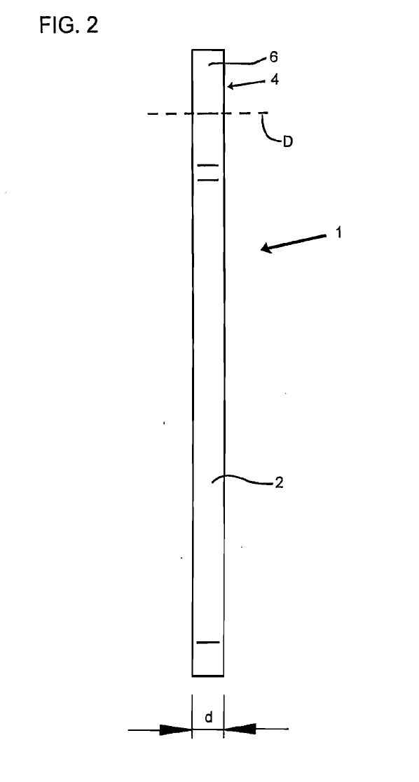

FIG. 2 a side view of the ratchet according to the invention pursuant to FIG. 1;

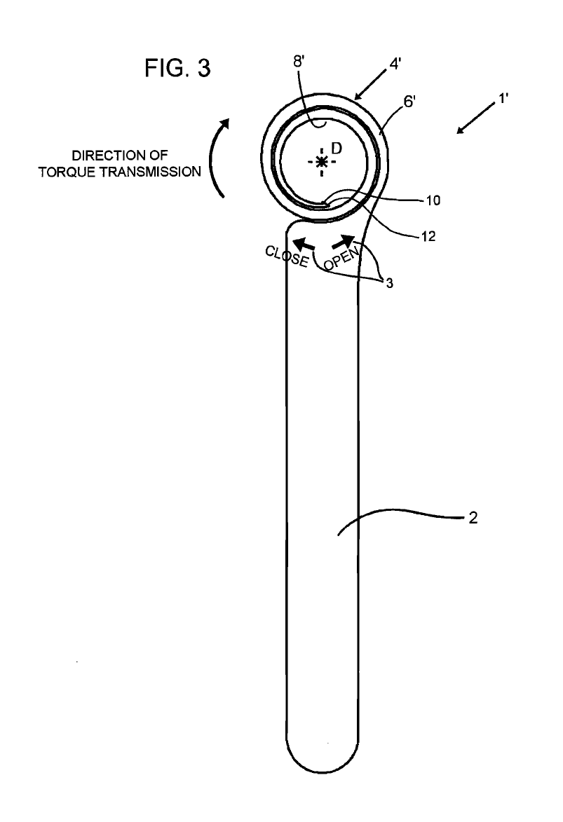

FIG. 3 a plan view of a ratchet according to the invention with a projection on a free end of a spiral;

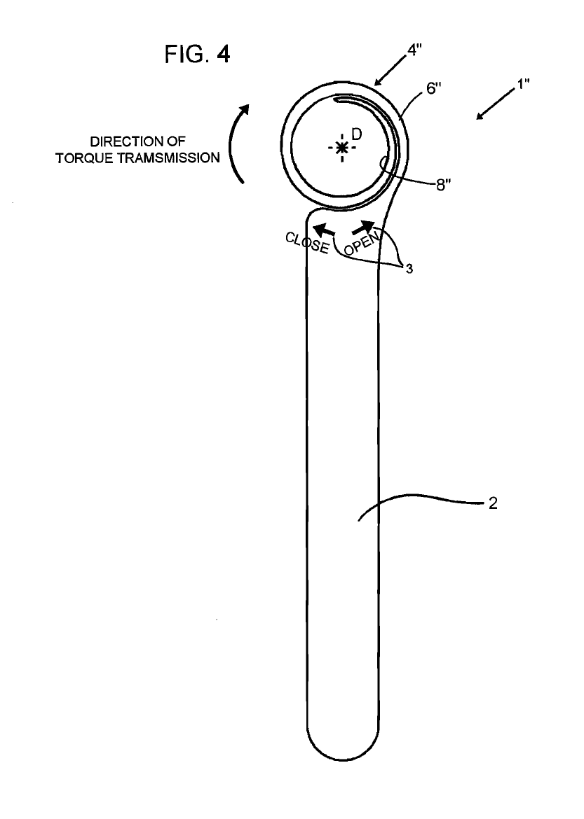

FIG. 4 a plan view of a ratchet according to the invention with a spiral having one and a half turns;

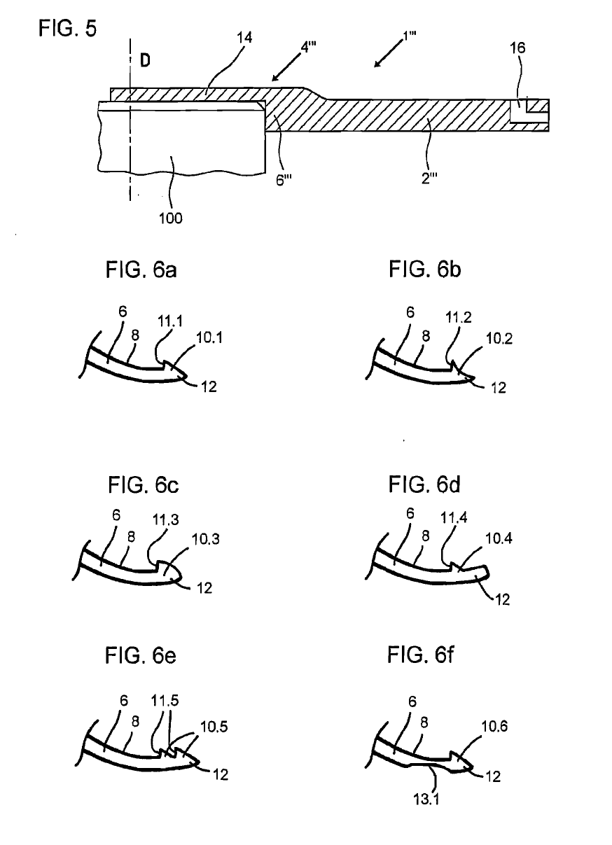

FIG. 5 a cross-section of a ratchet according to the invention with a stop;

FIG. 6 a)-f) a plurality of enlarged illustrations of the projection on the ratchet according to the invention;

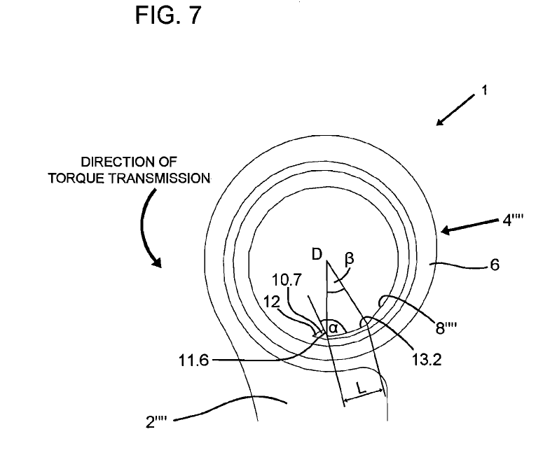

FIG. 7 a plan view of a ratchet according to the invention with a projection on a free end and an adjoining recess of a spiral;

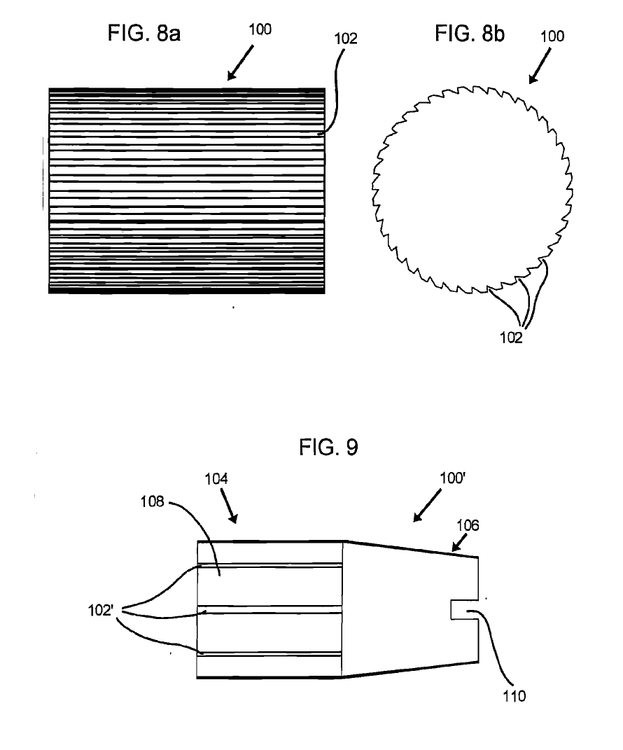

FIG. 8a a side view of an implant driver;

FIG. 8b a plan view of the implant driver pursuant to FIG. 7a; and

FIG. 9 a side view of a further implant driver.

FIG. 1 illustrates a plan view of a ratchet 1 according to a first exemplary embodiment of the present invention.

The ratchet 1 according to the first embodiment is of single-piece design and consists of a handle 2 and a pivot housing 4 having the same thickness d. The pivot housing 4 is designed as an archimedic spiral 6 with two turns. A pivot axis D with respect to which the torque transmission from the ratchet 1 to an implant driver 100 is performed is positioned in the pivot housing 4. It is noted that the arrow with the indicated direction of torque transmission relates to the rotation of the ratchet 1.

Since the spiral 6 is designed with two turns, the contact force on the implant driver 100 is also increased in that on rotation of the ratchet 1 in the direction of torque transmission a gap 5 available in a neutral position of the ratchet 1 and/or in the freewheeling direction does no longer exist, and hence edges of the spiral which get into contact with each other substantially support the torque transmission. These edges are also roughened to support the torque transmission.

An edge 8 of the spiral facing the pivot axis D is roughened, so that the torque transmission from the spiral 1 to the implant driver 100 can be performed more efficiently.

Furthermore, the edge 8 facing the pivot axis D is hardened so as to minimize or avoid wear with respect to the implant driver 100.

Markings 3 on the ratchet 1, here applied in the region of the handle 2, which indicate an open/closed direction, serve the user with respect to easy handling of the ratchet 1, so that injuries to the tissue or in the oral region can be prevented efficiently. Thus, the user can see clearly in which direction to actuate the ratchet 1 for torque transmission or for freewheeling, respectively. The markings 3 on the ratchet 1 may also be glued, etched, engraved, lasered or be formed by some other relief printing, gravure printing, screen printing, pad printing, porous printing or flat printing technique.

FIG. 2 illustrates a side view of the ratchet 1 according to FIG. 1 in which it can be recognized that the spiral 6 of the ratchet 1 is coiling up in a plane, i.e. two-dimensionally The thickness d of the ratchet is 2 mm.

FIG. 3 illustrates a plan view of a ratchet 1′ with a projection 10 in a second exemplary embodiment of the present invention. The projection 10 is arranged on a free end 12 of a spiral 6′ on an edge 8′ of the spiral 6′ facing the pivot axis D.

The ratchet 1′ according to the second embodiment is further designed in one piece and consists also of a handle 2 and a pivot housing 4′ having the same thickness d. The pivot axis D is positioned inside the pivot housing 4′. Similarly to FIG. 1 the arrow with the indicated direction of torque transmission relates to the rotation of the ratchet 1′.

FIG. 4 illustrates a plan view of a ratchet 1″ according to a third embodiment with a spiral 6″ having one and a half turns.

FIG. 5 illustrates a cross-section of a ratchet 1″′ with a stop 14 according to a further embodiment of the present invention. The stop 14 projecting from the pivot housing 4″′ and/or the handle 2″′ is designed such that it projects at least partially into the region of an implant driver receptacle, so that the ratchet 1″′ can be placed on an implant driver 100 only up to a predefined region. Thus, injuries of the tissue by too “deep” placing may be prevented efficiently. The handle 2″′ further comprises a receptacle 16 on the side facing away from the pivot housing 4″′. This receptacle 16 may serve to attach an extension of the handle 2″′ so as to reach, for instance, places that would be inaccessible otherwise.

FIG. 6 illustrates a plurality of enlarged views of the projection 10 on the spiral 6′ according to the ratchet 1′. In illustration 6a) a projection 10.1 on the end 12 of the spiral 6′ is of wedge-like design. The projection 10.1 is of such a wedge-shaped design that the wedge drops toward the end 12. By this wedge-like projection 10.1 it is possible to firmly arrest an implant driver 100 illustrated in FIGS. 8a and 8b in the direction of torque transmission and to release it again in a particularly good manner in freewheeling direction due to the wedge shape. In illustration 6b) a projection 10.2 is also positioned on the end 12. The projection 10.2 has an orthogonal face 11.2 with respect to the edge 8 which faces away from the end 12 and a concave design on the face facing the end 12. Instead of the concave design, a projection 10.3 is of convex design in illustration 6c). Illustration 6d) clarifies that the arrangement of a projection 10.4 may also be performed along the edge 8 facing the pivot axis D. Similarly, a plurality of projections 10.5, as becomes clear from illustration 6e), may also be arranged on the edge 8 facing the pivot axis D. The number of projections 10.5 is not restricted to two, as shown in illustration e). Furthermore, the projections 10.5 may also be arranged with a predetermined distance. Illustration 6b) shows a configuration as in illustration 6a) with the difference that the spiral 6 comprises a recess 13.1 on the face facing away from the pivot axis D. This recess 13.1 extends from the projection 10.6 over a predetermined length. This way it is possible to adjust the flexibility of the region of the projection.

These foregoing designs of the projection/the projections 10 make it possible to achieve a form-fit connection between the ratchet 1 and the implant driver 100.

FIG. 7 is a plan view of a ratchet 1″″ according to the invention with a projection 10.7 on a free end 12 and an adjoining recess 13.2 of a spiral 6″″.

The ratchet according to this fourth embodiment is formed of a handle 2″″ and a pivot housing 4″″ both having the same thickness d. The pivot housing 4″″ is formed as a spiral 6″″ having a wedge-shaped projection 10.7 on the free end 12 thereof. The projection 10.7 is of such a wedge-shaped design that the wedge drops toward the free end 12 of the spiral 6″″. Furthermore, the spiral 6″″ has a recess 13.2 extending along a length L. The projection 10.7 further comprises a face 11.6 facing away from the free end 12 of the spiral 6″″ which is defined by an angle α. The angle α extends from the face 11.6 facing away from the free end 12 to an edge 8 facing the pivot axis D.

The edge 8 facing the pivot axis D is provided with a recess 13.2 directly before the face facing away from the free end 12, said recess 13.2 extending over a predetermined length L. This recess 13.2 directly adjoins the face 11.6 of the projection 10.7 facing away from the free end 12 of the spiral 6″″. The transition between a continuous tapering and the recess 13.2 is formed continuously.

Although the length of the recess 13.2 of the spiral 6″″ is measured by a length L, it may of course also be measured in radian measure with the angle β, as results from FIG. 7. It is not required, either, that the recess 13.1 of the spiral 6″″ directly adjoins the face 11.6 facing away from the free end 12.

Furthermore, FIG. 7 illustrates that the cross-section of the spiral 6″″ itself tapers from the end arranged on the handle 2″″ to the free end 12. The additional forming of the recess 13.2 in the region of the free end 12, which is to be understood as a further additional tapering, results in that the spiral 6″″ bends back flexibly in the region of the recess 13.2, and hence also the projection 10.7, when too high a torque is transmitted, in other words, bends away from the implant driver 100. Depending on the design of the recess 13.2 and/or the spiral 6″″ a torque restriction may thus be provided in this region.

FIG. 8a illustrates a side view of an implant driver 100. FIG. 8b is a plan view of an implant driver 100 in which a plurality of recesses 102 have a division of 40. The recesses 102 are of wedge-shaped design, in other words, complementary to the recess 10 of the ratchet 1′ pursuant to FIG. 3. It may, however, also be of advantage if a ratchet 1″″ according to the fourth embodiment is used for torque transmission since the torque may then be restricted and/or adjusted on demand. The implant driver 100 is of cylindrical design and comprises a plurality of recesses 102 on a shell surface 108. The pitch of the wedge-like recesses 102 may be designed in due consideration of the ratchets, so that a desired torque transmission may be adjusted.

FIG. 9 illustrates a further implant driver 100′ divided into two sections 104 and 106. The first section 104 is of cylindrical design. A plurality of recesses 102′ are arranged along the shell surface 108 thereof The second section 106 is of frustoconical design and comprises at its end a receptacle 110 for a medical tool which is indicated schematically here.

With respect to the configuration of the handle 2 and the number thereof, reference is made to FIGS. 5a, 5b, 5c and 7a, 7b, 7c as well as to the associated description of the German patent application DE 10 2011 052 422.

In addition to the embodiments explained, the invention allows for further approaches of design.

The pivot axis of the pivot housing and a longitudinal axis of the handle may be spaced apart from each other. The pivot housing may thus be configured as an angle to the handle. With this advantageous design of the ratchet it is possible for the user to work at places difficult to access, especially in the region of the molars, due to the spacing apart of the pivot axis from the longitudinal axis. The pivot axis of the pivot housing and the longitudinal axis of the handle may also intersect.

The pivot axis of the pivot housing and the longitudinal axis of the handle may intersect at an angle of 30° to 150°, preferably of 60° to 120°, particularly preferred of 90°, relative to the longitudinal axis. This design has advantages for the placing of the ratchet on the implant driver, in particular in the case of regions that are difficult to access. It is readily possible to arrange a securable joint between the pivot housing and the handle, enabling a continuous adjustment of the angle on demand. The larger the angular dimension (arc length) of the spiral, the more pitch to the wrench is enabled.

A spring-like transition that is partially elastic and/or flexible at least vertically to the pivot axis D may, for instance, be formed between the handle 2 and the pivot housing 4.

The transition may be formed in a meandering pattern. This design is of advantage for the placing of the single-piece ratchet 1 on the implant driver 100.

Although the ratchet is of single-piece configuration in the foregoing embodiments, it may also be formed in several pieces.

It is further possible that an edge facing the pivot axis is formed to be smooth and the torque transmission is merely performed by one projection or by the spring force caused by the compressed spiral.

Depending on the design size, the thickness d of the ratchet 1 may optionally measure between 0.5 mm and 5 mm, preferably between 1 mm and 4 mm, particularly preferred 2.5 mm.

For better handling the handle may be serrated or structured. Although the handle is of cylindrical design, it may also have a triangular, square, rectangular cross-section or the like.

Although at least one projection may be formed in the spiral, it is also possible to provide at least one corresponding punctiform recess and to provide the implant driver with corresponding projections. It is further also conceivable to combine recesses and projections along the spiral.

Although the spiral coils up in a plane, a further developed spiral may coil up over several planes (three-dimensionally), so that the one edge of the spiral facing the pivot axis encloses the implant driver with an enclosing angle of more than 360°.

Although the recesses extend along the shell surface with a periodic distance, the recesses may also be formed aperiodically.

Although the projection is hardened, it may also be soft-annealed.

The recess 13.2 need not be available on the side of the spiral facing the pivot axis D, but may alternatively or in supplement also be arranged on the side and/or face of the spiral facing away from the pivot axis D. In a simplified design, this recess 13.2 may, however, also be omitted.

The torque to be transmitted may, for instance, also depend on the projection height in combination with the recess depth facing away from the pivot axis. During torque transmission this recess may allow sufficient clearance to the adjoining region and/or section of the spiral up to the projection and possibly to the projection itself so as to spring back, away from the implant driver, when a possibly too large torque is transmitted, and thus enable a torque restriction.

Although the recesses are formed on the face facing the pivot axis, they may also be formed on the front side of the ratchet. Thus, it is possible to use the ratchet more flexibly in the case of constricted space.

An alternative method for manufacturing the ratchet may be performed such that at first a blank in the form of a material block is selected. It may then be worked such that the desired contour of the ratchet is formed. Subsequently, the material block comprising the ratchet contour is separated into individual ratchets with a thickness d. Optionally, the separated ratchets may be further processed. In this respect, laser cutting, water jet cutting, wire eroding, spark eroding or punching may be used.

Although the ratchet 1 has been discussed for the medical field, in particular for the dental field, in the foregoing, the ratchet 1 may also be used in fields having a high demand to cleanness, such as, for instance, in space flight, in semiconductor manufacturing, or the like.

A ratchet is provided, in particular a ratchet for surgical interventions or a dental ratchet, wherein the ratchet has a handle and a pivot housing arranged thereon, said pivot housing being a flat spiral. The spiral is flexible and can be actuated such that the spiral coils up in a plane that is oriented substantially vertically to the pivot axis D. The invention also relates to a torque transmission system which comprises a ratchet and an implant driver. The implant driver has a first portion that is cylindrical and a second portion that is cylindrical or frustoconical, the second portion additionally having a receptacle for a medical tool, a surgical implant or any other object. In this way, an improved ratchet is obtained with which the torque to be applied can be applied to the implant driver in a metered manner.

1.-28. (canceled)

29. A ratchet for the medical field, wherein the rachet comprises a handle and a pivot housing arranged thereon, the pivot housing being a flat spiral which is flexible and designed as a logarithmic spiral, a hyperbolic spiral or an archimedic spiral, and wherein the ratchet can be actuated such that the spiral coils up in a plane that is oriented substantially vertically to a pivot axis (D).

30. The rachet of claim 29, wherein the rachet is of a single-piece design.

31. The rachet of claim 29, wherein the spiral comprises an angle of at least 360°.

32. The rachet of claim 29, wherein the spiral has at least one projection facing the pivot axis (D).

33. The ratchet of claim 32, wherein the spiral has a recess extending across a length (L), and wherein the recess directly adjoins a surface of the projection which faces away from a free end of the spiral.

34. The rachet of claim 29, wherein an edge of the spiral facing the pivot axis (D) is roughened in at least one section and/or is hardened in at least one section and/or wherein the spiral has at least one projection facing the pivot axis (D) and the projection is hardened in at least one section.

35. The rachet of claim 29, wherein a marking is provided in a region of the handle and/or the pivot housing.

36. The rachet of claim 29, wherein the rachet comprises a stop projecting at least partially into a region of a spiral center.

37. A torque transmission system, wherein the system comprises the ratchet of claim 29 and an implant driver, which implant driver comprises a first section of cylindrical design and a second section of cylindrical or frustoconical design, the second section further comprising a receptacle for a medical tool, a surgical implant, or any other object.

38. The torque transmission system of claim 37, wherein a force-fit connection exists between the ratchet and the implant driver.

39. The torque transmission system of claim 37, wherein a form-fit connection exists between the ratchet and the implant driver.

40. The torque transmission system of claim 39, wherein the first section of the implant driver comprises at least one recess arranged along a shell surface thereof, and wherein a force-fit connection is established by at least one projection of the spiral of the ratchet engaging with the at least one recess of the implant driver.

41. The torque transmission system of claim 40, wherein a plurality of recesses of the implant driver are arranged with periodic distances along the shell surface of the implant driver.

42. A method of transmitting a torque to an implant driver in surgery or in the dental field, wherein the method comprises using the rachet of claim 29 to transmit the torque.

43. A method of transmitting a torque to an implant driver by the ratchet of claim 29, wherein the method comprises:
(a) placing the ratchet on the implant driver in an axial direction thereof;
(b) fixing the ratchet on the implant driver and transmitting the torque to the implant driver by rotating the ratchet in a direction of torque transmission;
(c) releasing the implant driver by rotating the ratchet positioned thereon in freewheeling direction;
(d) detaching the ratchet from the implant driver by rotating in freewheeling direction while simultaneously removing the ratchet from the implant driver in the axial direction thereof.

44. The method of claim 43, wherein (b) and (c) are performed repeatedly before performing (d).

45. The method of claim 43, wherein (b) is performed such that at least one projection of a spiral of the ratchet is in engagement with at least one recess of the implant driver and (c) is performed such that the at least one projection of the spiral disengages from the at least one recess of the implant driver.

46. The method of claim 43, wherein (b) is performed such that a first part of the torque applied to the pivot housing of the ratchet continues to coil up the flexible spiral in a plane, so that a force-fit connection exists between an edge of the spiral facing a pivot axis (D) and the implant driver, and that a second part of the torque applied to the pivot housing causes a pivoting movement of the implant driver, and wherein (c) is performed such that the force-fit connection between the edge of the spiral facing the pivot axis (D) and the implant driver is released.

47. A method for manufacturing the ratchet of claim 29, wherein the method comprises providing a blank and forming a contour of the ratchet by laser cutting, water jet cutting, wire eroding, spark eroding, or punching.

48. The method of claim 47, wherein the blank has a uniform thickness (d).
